(19) United States
(12) Patent Application Publication
(10) Pub. No.: US 2011/0088711 A1
(43) Pub. Date: Apr. 21, 2011

(54) WIPE WITH AN EMULSION CONTAINING A THICKENING POLYMER AND A HYDROPHOBIC MODIFIED INULIN

(75) Inventor: Arnaud BONAFOS, Paris (FR)

(73) Assignee: L'OREAL, Paris (FR)

(21) Appl. No.: 12/819,257

(22) Filed: Jun. 21, 2010

Related U.S. Application Data
(60) Provisional application No. 61/224,565, filed on Jul. 10, 2009.
(60) Provisional application No. 61/224,088, filed on Jul. 9, 2009.

(30) Foreign Application Priority Data
Jun. 24, 2009 (FR) 09 54314
Jun. 24, 2009 (FR) 09 54318

(51) Int. Cl. B08B 1/00; A61K 8/92; A61Q 1/14

(52) U.S. Cl. 132/200; 510/136; 15/104.93

(57) ABSTRACT

The invention relates to an article, more particularly a wipe, having at least one water-insoluble substrate impregnated with a particular composition. The invention also relates to the composition itself and to the uses of the article in the cosmetic or dermatological field, in particular for cleaning and/or removing makeup from the skin. The article can in particular constitute a wipe which can be used especially for caring for the skin and/or hair and/or for cleaning and/or removing makeup from the skin.

### REFERENCE TO PRIOR APPLICATIONS

This application claims priority to U.S. provisional application Ser. Nos. 61/224,565, filed Jul. 10, 2009 and 61/224,088, filed Jul. 9, 2009; and to French patent applications 09 54318, filed Jun. 24, 2009 and 09 54314, filed Jun. 24, 2009, all incorporated herein by reference.

### FIELD OF THE INVENTION

The invention relates to an article, more particularly a wipe, comprising at least one water-insoluble substrate, and to a composition that can be impregnated, etc. on the substrate.

The invention also relates to the uses of the article in the cosmetic or dermatological field, in particular for cleaning and/or removing makeup from the skin.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. In this regard, the description herein is to be understood as illustrative in nature, and not as restrictive.

### BACKGROUND OF THE INVENTION

Cosmetic wipes are generally composed of a substrate made of a material of natural or synthetic origin, which is preferably a non-woven material but which can also be a foam or a woven material, the substrate being impregnated with a composition suitable for the desired objective, for example cleaning or removing makeup from the skin or eyes or also caring for the skin or hair. Such wipes are commonly used as they are valued for their practical side owing to the fact that they are disposable and that they are impregnated with the necessary and sufficient amount of treating or cleaning product. The use of these wipes avoids the handling and transportation of bottles comprising cosmetic compositions.

The impregnation of the substrates by the impregnating composition can be carried out according to different techniques, such as spraying or quenching. However, these techniques can be used only if the impregnating compositions are sufficiently fluid and have a viscosity close to that of water. This is because it is not possible to correctly wet the substrate when the compositions are too viscous as the substrate is then incorrectly impregnated and, furthermore, it is difficult to cut, fold and pack into bags. In addition, the article obtained is unpleasant to use as the impregnating product remains at the surface of the substrate or does not homogeneously impregnate it, with the result that some regions of the article comprise too much product while others are without it or comprise too little of it.

Furthermore, the fluid compositions which impregnate the wipes exhibit the disadvantage of comprising little oil and, for this reason, of having a care effect on the skin or a makeup-removing power which are regarded as inadequate. Furthermore, the wipes obtained exhibit a lack of comfort on application.

Thus, the need remains to have available a composition which exhibits a viscosity compatible with good impregnation of a support and which has an improved makeup-removing or cleaning effectiveness (via the incorporation of a higher level of oil) while being stable.

### DETAILED DESCRIPTION OF THE PREFERRED EMBODIMENTS

The inventor has found, surprisingly, that a composition in the form of an oil-in-water emulsion comprising a specific thickening/emulsifying polymer and an emulsifying agent which is an inulin modified by hydrophobic chains can be homogeneously impregnated on a wipe or any other water-insoluble substrate, making it possible to obtain the above desired properties.

Thus, a subject-matter of the present invention is an article comprising:

(A) a water-insoluble substrate comprising one or more layers, and

(B) a composition added to, in contact with, impregnated on, etc. the substrate, the composition being in the form of an oil-in-water emulsion comprising an oily phase dispersed in an aqueous phase, the emulsion further comprising:
at least one thickening polymer chosen from i) copolymers resulting from the polymerization of at least one monomer (a) chosen from carboxylic acids possessing α,β-ethylenic unsaturation or their esters with a monomer (b) possessing ethylenic unsaturation comprising a hydrophobic group, ii) polymers comprising at least one monomer possessing a sulpho group and their mixtures, and
at least one inulin modified by hydrophobic chains.

The term “article” is understood here to mean the combination composed of a water-insoluble support and a composition impregnated on the support. This article can in particular be a wipe but it can also have any form including those described below. The water-insoluble substrate is preferably absorbent and sufficiently strong not to disintegrate during the use thereof.

The article according to the invention is moist to the touch. It exhibits the advantage of being comfortable during application to the skin and having a nourishing effect due to the presence of an oily phase.

When it is used for cleaning or removing makeup from the skin, it is passed over the skin, while possibly leaving it applied for a time sufficient for the makeup products to be dissolved in the impregnating composition of the article, and then the skin is wiped. The skin can also optionally be rinsed subsequently.

The article according to the invention is preferably a cosmetic article appropriate for caring for and/or treating the skin of the face, body or hands and for cleaning or removing makeup from the skin of the face and/or body. It can also be used for caring for the hair and for removing makeup from the eyes.

The article according to the invention can have any form appropriate to the desired objective. It can constitute a wipe but it can also be in the form of a glove, of a mitten or in any other form appropriate for practical use on the face or the body, for example in the form of a face with holes for the sites of the eyes, nose and/or mouth, or in the form of a makeup-removing fingerstall for application in removing makeup from the eyelashes, or in the form of a single- or double-sided disc which can in particular comprise two sides impregnated with different compositions. The article can also comprise a rough surface which makes possible the exfoliation (scrubbing) of the skin.

Another subject-matter of the invention is a composition in the form of an oil-in-water emulsion comprising an oily phase dispersed in an aqueous phase, comprising:
at least one thickening polymer chosen from copolymers resulting from the polymerization of at least one monomer (a) chosen from carboxylic acids possessing α,β-ethylenic unsaturation or their esters with a monomer (b) possessing ethylenic unsaturation comprising a hydrophobic group, polymers comprising at least one monomer possessing a sulpho group and their mixtures, and
at least one inulin modified by hydrophobic chains,
the aqueous phase being present in a content of greater than or equal to 40% by weight, with respect to the total weight of the composition, and the composition comprising less than 5% by weight of additional emulsifying agents, with respect to the total weight of the composition.

Another subject-matter of the invention is the use of the article as defined above for caring for the skin or hair and/or cleaning and/or removing makeup from and/or scrubbing the skin.

The composition used according to the invention for impregnating the water-insoluble substrate, as it is intended for topical application, comprises a physiologically acceptable medium, that is to say a medium compatible with keratinous substances, such as the skin, mucous membranes, scalp or keratinous fibres, such as the eyelashes and hair.

The term “topical application” is understood here to mean external application to keratinous substances, which are in particular the skin, scalp, eyelashes, eyebrows, nails, mucous membranes and hair.

The composition conveyed by the article according to the invention preferably exhibits the advantage of being satisfactorily harmless and of having good cosmetic properties, that is to say a homogeneous texture which is pleasant on application, and also a satisfactory makeup-removing effectiveness by virtue of the presence of oils.

In addition, it is preferably very stable over time. An emulsion is stable if no change in its macroscopic or microscopic appearance and in its physicochemical characteristics (size of the drops, pH, viscosity) is observed after storing at ambient temperature or at 45° C. for a period of 2 months.

The composition of the invention exhibits a viscosity which can range, for example, from 1 to 120 mPa·s, preferably from 5 to 115 mPa·s, better still from 10 to 110 mPa·s and even better still from 50 to 100 mPa·s, at a temperature of 25° C., the viscosity being measured using a Rheomat 180 (Lamy) equipped with an MS-R1, MS-R2, MS-R3, MS-R4 or MS-R5 spindle, chosen according to the consistency of the composition, rotating at a rotational speed of 200 rev/min.

### Hydrophobic Modified Inulin

The term “hydrophobic modified inulin” according to the invention is understood to mean in particular an inulin modified by hydrophobic chains, especially modified by grafting hydrophobic chains to the hydrophilic backbone of the inulin.

Inulin belongs to the family of the fructans.

Fructans or fructosans are oligosaccharides or polysaccharides comprising a sequence of anhydrofructose units optionally in combination with one or more saccharide residues other than fructose. Fructans can be linear or branched. Fructans can be products obtained directly from a vegetable or microbial source or else products having a chain length which has been modified (increased or reduced) by fractionation, synthesis or hydrolysis, in particular by enzymes. Fructans generally have a degree of polymerization of 2 to approximately 1000 and preferably of 2 to approximately 60.

3 groups of fructans are distinguished. The first group corresponds to products having fructose units which are generally bonded via β-2-1 bonds. These are essentially linear fructans, such as inulins.

The second group also corresponds to linear fructoses but the fructose units are essentially bonded via β-2-6 bonds. These products are levans.

The third group corresponds to mixed fructans, that is to say having β-2-6 and β-2-1 sequences. These are essentially branched fructans, such as graminans.

Inulin can be obtained, for example, from chicory, dahlia or Jerusalem artichoke. Preferably, the inulin used in the composition according to the invention is obtained, for example, from chicory.

The inulins used in the compositions according to the invention are hydrophobic modified inulins. In particular, they are obtained by grafting hydrophobic chains to the hydrophilic backbone of the fructan.

The hydrophobic chains capable of being grafted to the main chain of the fructan can in particular be saturated or unsaturated and linear or branched hydrocarbon chains having from 1 to 50 carbon atoms, such as alkyl, arylalkyl, alkylaryl or alkylene groups, divalent cycloaliphatic groups or organopolysiloxane chains. These hydrocarbon or organopolysiloxane chains can in particular comprise one or more ester, amide, urethane, carbamate, thiocarbamate, urea, thiourea and/or sulphonamide functional groups, such as, in particular methylenedicyclohexyl and isophorone, or divalent aromatic groups, such as phenylene.

In particular, the inulin exhibits a degree of polymerization of 2 to approximately 1000, preferably of 2 to approximately 60, and a degree of substitution of less than 2, on the basis of one fructose unit.

According to a preferred embodiment, the hydrophobic chains exhibit at least one alkylcarbamate group of formula R—NH—CO— in which R is an alkyl group having from 1 to 22 carbon atoms.

According to a more preferred embodiment, the hydrophobic chains are laurylcarbamate groups.

In particular, mention may be made, by way of illustration and without implied limitation of the hydrophobic modified inulins which can be used in the compositions according to the invention, of stearoyl inulin, such as those sold under the names Lifidrem INST by Engelhard and Rheopearl INS by Ciba, palmitoyl inulin, undecylenoyl inulin, such as those sold under the names Lifidrem INUK and Lifidrem INUM by Engelhard, and inulin lauryl carbamate, such as that sold under the name Inutec SP1 by Orafti.

In particular, use is made of an inulin grafted with laurylcarbamate, in particular resulting from the reaction of lauryl isocyanate with an inulin, especially resulting from chicory. Mention may in particular be made, as example of these compounds, of the product sold under the name Inutec SP1 by Orafti.

The level of hydrophobic modified inulin in the composition of the invention can range from 0.01 to 20% by weight, preferably from 0.01 to 10% by weight, in particular from 0.05 to 10% by weight, preferably from 0.1 to 10% by weight, better still from 0.1 to 5% by weight and more preferably still from 0.1 to 1% by weight, with respect to the total weight of the composition.

### Thickening Polymer

The thickening (or gelling) polymer used in the composition of the invention is chosen from i) copolymers resulting from the polymerization of at least one monomer (a) chosen from carboxylic acids possessing α,β-ethylenic unsaturation or their esters with at least one monomer (b) possessing ethylenic unsaturation comprising a hydrophobic group, ii) polymers comprising at least one monomer possessing a sulpho group and their mixtures.

This polymer can also exhibit emulsifying properties.

The thickening polymer is preferably anionic.

The thickening polymer according to the invention can represent, as active material, from 0.01 to 10% by weight, preferably from 0.05 to 5% by weight, better still from 0.05 to 3% by weight and even better still from 0.05 to 1% by weight, with respect to the total weight of the composition.

i) Copolymer of at Least One Monomer (a) Chosen from Carboxylic Acids Possessing α,β-Ethylenic Unsaturation or Their Esters with at Least One Monomer (b) Possessing Ethylenic Unsaturation Comprising a Hydrophobic Group

The term “copolymers” is understood to mean both copolymers obtained from two types of monomers and those obtained from more than two types of monomers, such as terpolymers obtained from three types of monomers.

Their chemical structure more particularly comprises at least one hydrophilic unit and at least one hydrophobic unit. The term “hydrophobic group” or “hydrophobic unit” is intended to mean a radical possessing a saturated or unsaturated and linear or branched hydrocarbon chain comprising at least 8 carbon atoms, preferably from 10 to 30 carbon atoms, in particular from 12 to 30 carbon atoms and more preferably from 18 to 30 carbon atoms.

Preferably, the thickening polymers are chosen from copolymers resulting from the polymerization:
of at least one monomer of following formula (1):

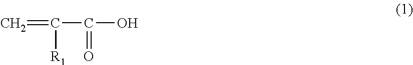

in which R₁ denotes H or CH₃ or C₂H₅, that is to say acrylic acid, methacrylic acid or ethacrylic acid monomers, and
of at least one monomer of unsaturated carboxylic acid (C₁₀-C₃₀)alkyl ester type which corresponds to the monomer of following formula (2):

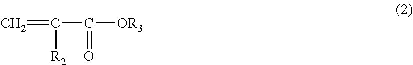

in which R₂ denotes H or CH₃ or C₂H₅ (that is to say, acrylate, methacrylate or ethacrylate units) and preferably H (acrylate units) or CH₃ (methacrylate units) and R₃ denotes a C₁₀-C₃₀ and preferably C₁₂-C₂₂ alkyl radical.

The (C₁₀-C₃₀) alkyl esters of unsaturated carboxylic acids are preferably chosen from lauryl acrylate, stearyl acrylate, decyl acrylate, isodecyl acrylate, dodecyl acrylate and the corresponding methacrylates, such as lauryl methacrylate, stearyl methacrylate, decyl methacrylate, isodecyl methacrylate and dodecyl methacrylate, and their mixtures.

According to a preferred embodiment, these thickening polymers are crosslinked.

Use will more particularly be made, among the thickening polymers of this type, of copolymers resulting from the polymerization of a mixture of monomers comprising:
(i) essentially acrylic acid,
(ii) an ester of formula (2) described above in which R₂ denotes H or CH₃ and R₃ denotes an alkyl radical having from 12 to 22 carbon atoms,
(iii) and a crosslinking agent which is a well-known copolymerizable polyethylenic unsaturated monomer, such as diallyl phthalate, allyl (meth)acrylate, divinylbenzene, (poly)ethylene glycol dimethacrylate and methylenebisacrylamide.

Use will more particularly be made, among copolymers of this type, of those composed of 95 to 60% by weight of acrylic acid (hydrophilic unit), 4 to 40% by weight of C₁₀-C₃₀ alkyl acrylate (hydrophobic unit) and 0 to 6% by weight of crosslinking polymerizable monomer or else of those composed of 98 to 96% by weight of acrylic acid (hydrophilic unit), 1 to 4% by weight of C₁₀-C₃₀ alkyl acrylate (hydrophobic unit) and 0.1 to 0.6% by weight of crosslinking polymerizable monomer, such as those described above.

Preference is very particularly given according to the present invention, among the polymers above, to acrylate/C₁₀-C₃₀ alkyl acrylate copolymers (INCI name: Acrylates/C10-30 Alkyl Acrylate Crosspolymer), such as the products sold by Lubrizol under the trade names Pemulen TR1, Pemulen TR2, Carbopol 1382 and Carbopol EDT 2020 and more preferably still Pemulen TR-2.

### ii) Polymer Comprising at Least One Monomer Possessing a Sulpho Group

The thickening polymers comprising at least one monomer possessing a sulpho group used in the composition of the invention are soluble or dispersible or swellable in water. The polymers used in accordance with the invention can be homopolymers or copolymers and are capable of being obtained from at least one monomer possessing ethylenic unsaturation and possessing a sulpho group which can be in the free form or in the partially or completely neutralized form. These polymers can optionally comprise at least one hydrophobic group and can then constitute an amphiphilic polymer (or hydrophobic modified polymer).

Preferably, the polymers in accordance with the invention can be partially or completely neutralized by an inorganic base (sodium hydroxide, potassium hydroxide, aqueous ammonia) or an organic base, such as mono-, di- or triethanolamine, an aminomethylpropanediol, N-methylglucamine, basic amino acids, such as arginine and lysine, and the mixtures of these compounds. They are generally neutralized. The term “neutralized” is understood to mean, in the present invention, polymers which are completely or virtually completely neutralized, that is to say neutralized to at least 90%.

The polymers used in the composition of the invention generally have a number-average molecular weight ranging from 1000 to 20 000 000 g/mol, preferably from 20 000 to 5 000 000 g/mol and more preferably still from 100 000 to 1 500 000 g/mol.

These polymers according to the invention may or may not be crosslinked.

The monomers possessing a sulpho group of the polymer used in the composition of the invention are chosen in particular from vinylsulphonic acid, styrenesulphonic acid, (meth)acrylamido(C₁-C₂₂)alkylsulphonic acids, N—(C₁-C₂₂)alkyl(meth)acrylamido(C₁-C₂₂)alkylsulphonic acids, such as undecylacrylamidomethanesulphonic acid, and also their partially or completely neutralized forms, and their mixtures.

According to a preferred embodiment of the invention, the monomers possessing a sulpho group are chosen from (meth) acrylamido (C₁-C₂₂) alkylsulphonic acids, such as, for example, acrylamidomethanesulphonic acid, acrylamidoethanesulphonic acid, acrylamidopropanesulphonic acid, 2-acrylamido-2-methylpropanesulphonic acid, 2-methacrylamido-2-methylpropanesulphonic acid, 2-acrylamido-n-butanesulphonic acid, 2-acrylamido-2,4,4-trimethylpentanesulphonic acid, 2-methacrylamidododecylsulphonic acid, 2-acrylamido-2,6-dimethyl-3-heptanesulphonic acid, and also their partially or completely neutralized forms, and their mixtures.

Use is more particularly made of 2-acrylamido-2-methylpropanesulphonic acid (AMPS) and of its partially or completely neutralized forms.

When the polymers are crosslinked, the crosslinking agents can be chosen from the compounds possessing polyolefinic unsaturation commonly used for the crosslinking of polymers obtained by radical polymerization.

Mention may be made, for example, as crosslinking agents, of divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allyl ethers of alcohols of the series of the sugars, or other allyl or vinyl ethers of polyfunctional alcohols, and allyl esters of phosphoric and/or vinylphosphonic acid derivatives, or the mixtures of these compounds.

According to a preferred embodiment of the invention, the crosslinking agent is chosen from methylenebisacrylamide, allyl methacrylate or trimethylolpropane triacrylate (TMPTA). The degree of crosslinking generally ranges from 0.01 to 10 mol % and more particularly from 0.2 to 2 mol %, with respect to the polymer.

When the polymers used are homopolymers, they comprise only monomers possessing a sulpho group and, if they are crosslinked, one or more crosslinking agents.

The preferred AMPS homopolymers are generally wherein they comprise, distributed randomly:

a) from 90 to 99.9% by weight of units of following general formula (II):

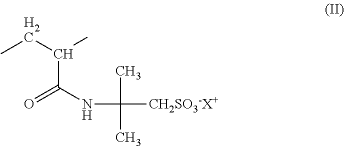

in which X⁺ denotes a proton, an alkali metal cation, an alkaline earth metal cation or the ammonium ion, it being possible for at most 10 mol % of the cations X⁺ to be protons H⁺;

b) from 0.01 to 10% by weight of crosslinking units originating from at least one monomer having at least two olefinic double bonds; the proportions by weight being defined with respect to the total weight of the polymer.

The homopolymers according to the invention which are more particularly preferred comprise from 98 to 99.5% by weight of units of formula (II) and from 0.2 to 2% by weight of crosslinking units.

Mention may in particular be made, as polymers of this type, of the crosslinked and neutralized homopolymer of 2-acrylamido-2-methylpropanesulphonic acid sold by Clariant under the trade name “Hostacerin AMPS” (CTFA name: ammonium polyacryldimethyltauramide).

The polymer can also be an amphiphilic homopolymer (or hydrophobic modified homopolymer) chosen from random amphiphilic AMPS polymers modified by reaction with a mono(C₆-C₂₂ n-alkyl)amine or a di(C₆-C₂₂ n-alkyl)-amine, such as those described in the document WO-A-00/31154, which are grafted homopolymers.

When the polymers used are copolymers, they are capable of being obtained from monomers possessing ethylenic unsaturation and possessing a sulpho group and from other monomers possessing ethylenic unsaturation, that is to say from monomers possessing ethylenic unsaturation without a sulpho group.

The monomers possessing ethylenic unsaturation and possessing a sulpho group are chosen from those described above.

The monomers possessing ethylenic unsaturation without a sulpho group can be chosen from hydrophilic monomers possessing ethylenic unsaturation, hydrophobic monomers possessing ethylenic unsaturation and their mixtures. When the polymer comprises hydrophobic monomers, it constitutes an amphiphilic polymer (also known as hydrophobic modified polymer).

The hydrophilic monomers possessing ethylenic unsaturation can be chosen, for example, from (meth)acrylic acids, their alkyl derivatives substituted at the β position or their esters obtained with monoalcohols or mono- or polyalkylene glycols, (meth)acrylamides, vinylpyrrolidone, vinylformamide, maleic anhydride, itaconic acid, maleic acid or the mixtures of these compounds.

When the polymer of the composition according to the invention is a copolymer capable of being obtained from monomers possessing ethylenic unsaturation and possessing a sulpho group and from hydrophilic monomers possessing ethylenic unsaturation, it can be chosen in particular from (1) crosslinked anionic copolymers of acrylamide or methacrylamide and of 2-acrylamido-2-methylpropanesulphonic acid, in particular those which are provided in the form of a W/O emulsion, such as those sold under the name of Sepigel 305 by Seppic (CTFA name: Polyacrylamide/C13-14 Isoparaffin/Laureth-7) or under the name of Simulgel 600 by Seppic (CTFA name: Acrylamide/Sodium acryloyldimethyltaurate copolymer/Isohexadecane/Polysorbate 80), (2) copolymers of (meth)acrylic acid or of (meth)acrylate and of 2-acrylamido-2-methylpropanesulphonic acid, in particular those which are provided in the form of a W/O emulsion, such as those sold under the name of Simulgel NS by Seppic (sodium 2-acrylamido-2-methylpropanesulphonate/hydroxyethyl acrylate copolymer as a 40% inverse emulsion in Polysorbate 60 and squalane) (CTFA name: hydroxyethyl acrylate/sodium acryloyldimethyltaurate copolymer/squalane/polysorbate 60) or under the name of Simulgel EG by Seppic (acrylic acid/2-acrylamido-2-methylpropanesulphonic acid in the form of a sodium salt copolymer as a 45% inverse emulsion in isohexadecane/water) (CTFA name: Sodium Acrylate/Sodium Acryloyldimethyltaurate Copolymer/Isohexadecane/Polysorbate 80), and (3) copolymers of 2-acrylamido-2-methylpropanesulphonic acid and of vinylpyrrolidone or of vinylformamide, such as the products sold under the Aristoflex AVC names by Clariant.

When the monomers possessing a sulpho group are copolymerized with hydrophobic monomers possessing ethylenic unsaturation and comprising a hydrophobic chain, also known as fatty chain (C₆-C₅₀ chain), the polymer obtained is amphiphilic, that is to say that it comprises both a hydrophilic part and a hydrophobic part. Such polymers are also known as hydrophobic modified polymers.

These hydrophobic modified polymers can additionally comprise one or more monomers comprising neither a sulpho group nor a fatty chain, such as (meth)acrylic acids, their alkyl derivatives substituted at the β position or their esters obtained with monoalcohols or mono- or polyalkylene glycols, (meth)acrylamides, vinylpyrrolidone, vinylformamide, maleic anhydride, itaconic acid, maleic acid or the mixtures of these compounds.

Use may in particular be made, as hydrophobic modified polymers, of those capable of being obtained from 2-acrylamido-2-methylpropanesulphonic acid (AMPS) and from at least one hydrophobic monomer possessing ethylenic unsaturation and comprising at least one group having from 6 to 50 carbon atoms, more preferably from 6 to 22 carbon atoms, more preferably still from 6 to 18 carbon atoms and more particularly from 12 to 18 carbon atoms.

These polymers are described in particular in the documents EP-A-750899, U.S. Pat. No. 5,089,578 and WO-A-2002/43689 and in the following publications by Yotaro Morishima:
“Self-assembling amphiphilic polyelectrolytes and their nanostructures—Chinese Journal of Polymer Science”, Vol. 18, No. 40 (2000), 323-336;
“Micelle formation of random copolymers of sodium 2-(acrylamido)-2-methylpropanesulphonate and a non-ionic surfactant macromonomer in water as studied by fluorescence and dynamic light scattering”, Macromolecules 2000, Vol. 33, No. 10, 3694-3704;
“Solution properties of micelle networks formed by non-ionic moieties covalently bound to a polyelectrolyte: salt effects on rheological behaviour”, Langmuir, 2000, Vol. 16, No. 12, 5324-5332;
“Stimuli responsive amphiphilic copolymers of sodium 2-(acrylamido)-2-methylpropanesulphonate and associative macromonomers”, Polym. Preprint, Div. Polym. Chem., 1999, 40(2), 220-221.

The hydrophobic monomers of these specific polymers are preferably chosen from acrylates, alkyl acrylates, acrylamides or alkylacrylamides of following formula (III):

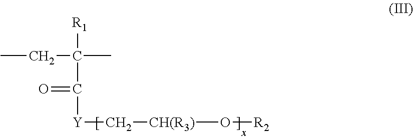

in which R₁ and R₃, which are identical or different, denote a hydrogen atom or a substantially linear or branched C₁-C₆ alkyl radical (preferably a methyl radical); Y denotes O or NH; R₂ denotes a hydrocarbon radical comprising from 6 to 50 carbon atoms, more preferably from 6 to 22 carbon atoms, more preferably still from 6 to 18 carbon atoms and more particularly from 12 to 18 carbon atoms; and x denotes a number of moles of alkylene oxide and varies from 0 to 100.

The R₂ radical is preferably chosen from substantially linear C₆-C₁₈ alkyl radicals (for example n-hexyl, n-octyl, n-decyl, n-hexadecyl, n-dodecyl or lauryl, or n-octadecyl or stearyl) or branched or cyclic C₆-C₁₈ alkyl radicals (for example cyclododecane (C₁₂) or adamantane (C₁₀)); perfluorinated C₆-C₁₈ alkyl radicals (for example the group of formula —(CH₂)₂—(CF₂)₉—CF₃); the cholesteryl radical (C₂₇) or a cholesterol ester residue, such as the cholesteryl oxyhexanoate group; or polycyclic aromatic groups, such as naphthalene or pyrene. Preference is more particularly given, among these radicals, to substantially linear alkyl radicals and more particularly to the n-dodecyl, n-hexadecyl or n-octadecyl radical, and to their mixtures.

According to a particularly preferred form of the invention, the monomer of formula (III) comprises at least one alkylene oxide unit (x≧1) and preferably several alkylene oxide units (x>1) constituting a polyoxyalkylene chain. The polyoxyalkylene chain is preferably composed of ethylene oxide units and/or of propylene oxide units and more particularly still is composed of ethylene oxide units. The number of oxyalkylene units (or number of moles of alkylene oxide) generally varies from 3 to 100, more preferably from 3 to 50 and more preferably still from 7 to 25.

Mention may be made, among these polymers, of:
crosslinked or non-crosslinked and neutralized or non-neutralized copolymers comprising from 15 to 60% by weight of AMPS units and from 40 to 85% by weight of (C₈-C₁₆)alkyl(meth)acrylamide units or of (C₈-C₁₆)alkyl (meth)acrylate units, with respect to the polymer, such as those described in the document EP-A-750 899;
terpolymers comprising from 10 to 90 mol % of acrylamide units, from 0.1 to 10 mol % of AMPS units and from 5 to 80 mol % of n-(C₆-C₁₀alkylacrylamide units, with respect to the polymer, such as those described in the document U.S. Pat. No. 5,089,578;
non-crosslinked copolymers of partially or completely neutralized AMPS and of n-dodecyl, n-hexadecyl or n-octadecyl methacrylate, such as those described in the abovementioned papers by Morishima;
crosslinked or non-crosslinked copolymers of partially or completely neutralized AMPS and of n-dodecylmethacrylamide, such as those described in the abovementioned papers by Morishima.

Mention may more particularly be made, as hydrophobic modified polymers, of the copolymers composed (i) of 2-acrylamido-2-methylpropanesulphonic acid (AMPS) units of formula (II) indicated above, in which X′ is a proton, an alkali metal cation, an alkaline earth metal cation or the ammonium ion, and (ii) of units of following formula (IV):

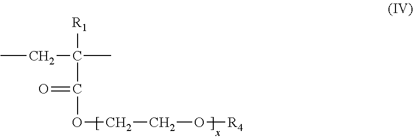

in which x denotes an integer varying from 3 to 100, preferably from 3 to 50 and more preferably from 7 to 25; R₁ has the same meaning as that indicated above in the formula (III) and R₄ denotes a linear or branched alkyl radical comprising from 6 to 22 carbon atoms and preferably from 10 to 22 carbon atoms.

The hydrophobic modified polymers of this type are in particular those described in the above-mentioned papers by Morishima, for which x=25, R₁ denotes methyl and R₄ represents n-dodecyl, or those described in the document WO-A-02/43689, for which x=8 or 25, R₁ denotes methyl and R₄ represents n-hexadecyl (C₁₆), n-octadecyl (C₁₈) or n-dodecyl (C₁₂), or their mixtures. The polymers for which X⁺ denotes sodium or ammonium are more particularly preferred.

The preferred hydrophobic modified polymers which can be used in the composition in accordance with the invention can be obtained according to conventional radical polymerization processes in the presence of one or more initiators, such as, for example, azobisisobutyronitrile (AIBN), azobisdimethylvaleronitrile, 2,2′-azobis[2-amidinopropane] hydrochloride (ABAH), organic peroxides, such as dilauryl peroxide, benzoyl peroxide, tert-butyl hydroperoxide, and the like, inorganic peroxide compounds, such as potassium persulphate or ammonium persulphate, or H₂O₂, optionally in the presence of reducing agents.

These hydrophobic modified polymers can be obtained in particular by radical polymerization in a tert-butanol medium, from which they precipitate. It is possible, using polymerization by precipitation from tert-butanol, to obtain a distribution in the size of the particles of the polymer which is particularly favourable to the uses thereof.

The reaction can be carried out at a temperature of between 0 and 150° C., preferably between 10 and 100° C., either at atmospheric pressure or under reduced pressure. It can also be carried out under an inert atmosphere and preferably under nitrogen.

These preferred hydrophobic modified polymers are in particular those described in the document EP-1 069 142 and especially those obtained by polymerization of 2-acrylamido-2-methylpropanesulphonic acid (AMPS) or one of its sodium or ammonium salts with an ester of (meth)acrylic acid and
of a C₁₀-C₁₈ alcohol oxyethylenated with 8 mol of ethylene oxide (Genapol® C-080 from Clariant),
of a C₁₁ oxo alcohol oxyethylenated with 8 mol of ethylene oxide (Genapol® UD-080 from Clariant),
of a C₁₁ oxo alcohol oxyethylenated with 7 mol of ethylene oxide (Genapol® UD-070 from Clariant),
of a C₁₂-C₁₄ alcohol oxyethylenated with 7 mol of ethylene oxide (Genapol® LA-070 from Clariant),
of a C₁₂-C₁₄ alcohol oxyethylenated with 9 mol of ethylene oxide (Genapol® LA-090 from Clariant),
of a C₁₂-C₁₄ alcohol oxyethylenated with 11 mol of ethylene oxide (Genapol® LA-110 from Clariant),
of a C₁₆-C₁₈ alcohol oxyethylenated with 8 mol of ethylene oxide (Genapol® T-080 from Clariant),
of a C₁₆-C₁₈ alcohol oxyethylenated with 15 mol of ethylene oxide (Genapol® T-150 from Clariant),
of a C₁₆-C₁₈ alcohol oxyethylenated with 11 mol of ethylene oxide (Genapol® T-110 from Clariant),
of a C₁₆-C₁₈ alcohol oxyethylenated with 20 mol of ethylene oxide (Genapol® T-200 from Clariant),
of a C₁₆-C₁₈ alcohol oxyethylenated with 25 mol of ethylene oxide (Genapol® T-250 from Clariant),
of a C₁₈-C₂₂ alcohol oxyethylenated with 25 mol of ethylene oxide and/or of an iso-C₁₆-C₁₈ alcohol oxyethylenated with 25 mol of ethylene oxide.

The mol % concentration of the units of formula (II) and of the units of formula (IV) in the polymers according to the invention varies according to the cosmetic application desired and the rheological properties desired for the formulation. It can vary between 0.1 and 99.9 mol %.

Preferably, for the most hydrophobic polymers, the molar proportion of units of formula (II) or (IV) varies from 50.1 to 99.9%, more particularly from 70 to 95% and more particularly still from 80 to 90%.

Preferably, for the polymers which are not very hydrophobic, the molar proportion of units of formula (II) or (IV) varies from 0.1 to 50%, more particularly from 5 to 25% and more particularly still from 10 to 20%.

The distribution of the monomers in the polymers of the invention can be, for example, alternating, block (including multiblock) or random.

Mention may in particular be made, as hydrophobic modified polymers of this type, of the copolymer of AMPS and of ethoxylated C₁₂-C₁₄ alkyl methacrylate (noncrosslinked copolymer obtained from Genapol LA-070 and from AMPS) (CTFA name: Ammonium Acryloyldimethyltaurate/Laureth-7 Methacrylate Copolymer) sold under the name Aristoflex LNC by Clariant, and the copolymer of AMPS and of ethoxylated (25 EO) stearyl methacrylate (copolymer crosslinked with trimethylolpropane triacrylate obtained from Genapol T-250 and from AMPS) (CTFA name: Ammonium Acryloyldimethyltaurate/Steareth-Methacrylate Crosspolymer) sold under the name Aristoflex HMS by Clariant.

Preferably, the polymer possessing a sulpho group is chosen from AMPS copolymers, preferably chosen from copolymers of (meth)acrylic acid or of (meth)acrylate and of 2-acrylamido-2-methylpropanesulphonic acid, in particular copolymers of alkyl acrylate and of AMPS, preferably copolymers of hydroxyalkyl acrylate and of AMPS.

Use may in particular be made of those which are provided in the form of a W/O emulsion, such as those sold under the name of Simulgel NS by Seppic (sodium 2-acrylamido-2-methylpropanesulphonate/hydroxyethyl acrylate copolymer as a 40% inverse emulsion in Polysorbate 60 and squalane) (CTFA name: Hydroxyethyl Acrylate/Sodium Acryloyldimethyltaurate Copolymer/Squalane/Polysorbate 60) or Simulgel FS by Seppic (sodium 2-acrylamido-2-methylpropanesulphonate/hydroxyethyl acrylate copolymer as a 40% inverse emulsion in Polysorbate 60 and isohexadecane) (CTFA name: Hydroxyethyl Acrylate/Sodium Acryloyldimethyltaurate Copolymer/Isohexadecane/Polysorbate 60) or those sold under the name of Simulgel EG by Seppic (acrylic acid/2-acrylamido-2-methylpropanesulphonic acid in the form of a sodium salt copolymer as a 45% inverse emulsion in isohexadecane/water) (CTFA name: Sodium Acrylate/Sodium Acryloydimethyltaurate Copolymer/Isohexadecane/Polysorbate 80).

According to one embodiment, the hydrophobic modified inulin and the thickening polymer as defined above are present in a hydrophobic modified inulin/thickening polymer ratio by weight ranging from 5:1 to 1:5, in particular from 4:1 to 1:4, preferably from 3:1 to 1:3 and more preferably from 3:1 to 1:1.

Oily Phase

The oily phase is a fatty phase comprising at least one fatty substance chosen from fatty substances which are liquid at ambient temperature (20-25° C.) or volatile or non-volatile oils of vegetable, mineral or synthetic origin, and their mixtures. These oils are physiologically acceptable.

The oily phase can also comprise any normal fat-soluble or fat-dispersible additive as indicated below.

They can in particular comprise other fatty substances, such as waxes, pasty compounds, fatty alcohols or fatty acids.

Mention may be made, as oils which can be used in the composition of the invention, for example, of:
hydrocarbon oils of animal origin, such as perhydrosqualene;
hydrocarbon oils of vegetable origin, such as liquid triglycerides of fatty acids comprising from 4 to 10 carbon atoms, such as the triglycerides of heptanoic acid or octanoic acid or also, for example, sunflower, maize, soybean, cucumber, grape seed, sesame, hazelnut, apricot, macadamia, arara, coriander, castor or avocado oils, triglycerides of caprylic/capric acids, such as those sold by Stearineries Dubois or those sold under the names Miglyol 810, 812 and 818 by Dynamit Nobel, jojoba oil or shea butter oil;
synthetic esters and ethers, in particular of fatty acids, such as oils of formulae R¹COOR² and R¹OR² in which R¹ represents the residue of a fatty acid or of a fatty alcohol comprising from 8 to 29 carbon atoms and R² represents a branched or unbranched hydrocarbon chain comprising from 3 to 30 carbon atoms, such as, for example, Purcellin oil, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate, triisocetyl citrate or heptanoates, octanoates or decanoates of fatty alcohols; polyol esters, such as propylene glycol dioctanoate, neopentyl glycol diheptanoate and diethylene glycol diisononanoate; and pentaerythritol esters, such as pentaerythrityl tetraisostearate;
linear or branched hydrocarbons of mineral or synthetic origin, such as volatile or non-volatile liquid paraffins and their derivatives, liquid petrolatum, polydecenes, isohexadecane, isododecane or hydrogenated polyisobutene, such as Parleam® oil;
fluorinated oils which are partially hydrocarbon-comprising and/or silicone-comprising, such as those described in the document JP-A-2-295912;
silicone oils, such as volatile or non-volatile polymethylsiloxanes (PDMSs) comprising a linear or cyclic silicone chain which are liquid or pasty at ambient temperature, in particular volatile silicone oils, especially cyclopolydimethylsiloxanes (cyclomethicones), such as cyclohexadimethylsiloxane and cyclopentadimethylsiloxane; polydimethylsiloxanes comprising pendant alkyl, alkoxy or phenyl groups or alkyl, alkoxy or phenyl groups at the end of the silicone chain, which groups have from 2 to 24 carbon atoms; or phenylated silicones, such as phenyl trimethicones, phenyl dimethicones, phenyl(trimethylsiloxy) diphenylsiloxanes, diphenyl dimethicones, diphenyl(methyldiphenyl)trisiloxanes, (2-phenylethyl)-trimethylsiloxysilicates and polymethylphenylsiloxanes;
their mixtures.

Mention may in particular be made of the oils chosen from:
the esters resulting from the reaction of at least one fatty acid comprising at least 6 carbon atoms, preferably from 6 to 26 carbon atoms, better still from 6 to 20 carbon atoms and even better still from 6 to 16 carbon atoms and of at least one alcohol comprising from 1 to 17 carbon atoms and better still from 3 to 15 carbon atoms,
ethers of a fatty acid comprising from 6 to 20 carbon atoms, such as dicaprylyl ether,
glycerol ethers comprising from 6 to 12 carbon atoms,

and their mixtures.

These esters can in particular be chosen from:

(1) the esters obtained from at least one aliphatic fatty acid possessing an unsaturated linear chain comprising at least 6 carbon atoms, preferably from 6 to 12 carbon atoms, and from at least one aliphatic alcohol possessing an unsaturated branched chain comprising from 2 to 12 carbon atoms,

(2) the esters obtained from at least one aliphatic fatty acid possessing an unsaturated linear chain comprising at least 6 carbon atoms, for example comprising from 8 to 17 carbon atoms, and from an aliphatic alcohol possessing an unsaturated linear chain comprising from 1 to 10 carbon atoms, preferably from 2 to 10 carbon atoms,

and their mixtures.

The esters are preferably mono- or diesters. Preferably, the esters do not comprise any unsaturation and/or any ether or hydroxyl group. More advantageously still, the ester is a saturated ester which does not include any ether or hydroxyl group.

Thus, the ester used as makeup-removing oil of the composition in accordance with the invention can be chosen in particular from isopropyl myristate, isopropyl palmitate, 2-ethylhexyl caprate/caprylate (or octyl caprate/caprylate), 2-ethylhexyl palmitate, isostearyl neopentanoate, isononyl isononanoate, hexyl laurate, esters of lactic acid and of fatty alcohols comprising 12 or 13 carbon atoms, dicaprylyl carbonate and their mixtures.

Mention may in particular be made, as ether which can be used in the composition according to the invention, of dicaprylyl ether (Cetiol OE from Cognis) or the 2-ethylhexyl ether of glycerol (INCI name: ethylhexylglycerin), such as Sensiva SC 50 from Schulke & Mayr GmbH.

Preferably, the composition comprises at least one oil chosen from isopropyl myristate, 2-ethylhexyl monococoate, isodecyl monopentanoate, dicaprylyl carbonate, isopropyl palmitate, 2-ethylhexyl palmitate, isononyl isononanoate and their mixtures.

The amount of oily phase can range, for example, from 2 to 35% by weight, preferably from 5 to 33% by weight, better still from 10 to 33% by weight and even better still from 15 to 30% by weight, with respect to the total weight of the composition.

This amount of oily phase does not comprise the amount of emulsifier or of thickening polymer.

In particular, the composition according to the invention comprises from 1 to 40% by weight of oils, with respect to the total weight of the composition, preferably from 2 to 30% by weight and better still from 5 to 25% by weight. According to one embodiment, the composition comprises at least 1% of oils, preferably at least 2% of oils, better still at least 5% of oils, even better still at least 10% of oils and more preferably at least 15% of oils, with respect to the total weight of the composition.

Aqueous Phase

The aqueous phase of the composition according to the invention comprises water and optionally one or more compounds which are miscible with water or at least partially miscible with water, such as polyols or lower C₂ to C₈ monoalcohols, such as ethanol and isopropanol. The term “ambient temperature” should be understood as meaning a temperature of approximately 25° C., at standard atmospheric pressure (760 mmHg).

The term “polyol” should be understood as meaning any organic molecule comprising at least two free hydroxyl groups. Mention may be made, as polyols, for example, of glycols, such as butylene glycol, propylene glycol, isoprene glycol, glycerol and polyethylene glycols, such as PEG-8, sorbitol or sugars, such as glucose.

The aqueous phase can also comprise any normal water-soluble or water-dispersible additive as indicated below.

Preferably, the aqueous phase represents at least 40% by weight, with respect to the total weight of the composition, preferably at least 50% by weight and better still at least 60% by weight.

The aqueous phase can represent from 60 to 98% by weight, preferably from 65 to 95% by weight, better still from 70 to 90% by weight and even better still from 70 to 85% by weight, with respect to the total weight of the composition.

Emulsifiers

The composition according to the invention can comprise one or more additional emulsifying agents (distinct from the thickening copolymer and the hydrophobic modified inulin).

However, according to one embodiment, the composition comprises less than 5% by weight, preferably less than 1% by weight, preferably less than 0.5% by weight, as active material, of additional emulsifying agents (emulsifying surfactants or emulsifying polymers) and/or additional thickening polymers, other than the thickening polymer and the hydrophobic modified inulin defined above. More preferably, the composition is devoid of additional emulsifying agents and/or additional thickening polymers.

Additives

In a known way, the composition for topical application of the invention can also comprise one or more of the adjuvants normal in the cosmetic or dermatological field. Mention may be made, as adjuvants, for example, of gelling agents, active principles, preservatives, antioxidants, fragrances, solvents, salts, fillers, sunscreens (=UV screening agents), colouring materials, basic agents (triethanolamine, diethanolamine, sodium hydroxide) or acidic agents (citric acid), and also lipid vesicles or any other type of vector (nanocapsules, microcapsules, and the like), and their mixtures. These adjuvants are used in the proportions usual in the cosmetic field, for example from 0.01 to 30% of the total weight of the composition, and they are, depending on their nature, introduced into the aqueous phase of the composition or into the oily phase, or also into vesicles or any other type of vector. These adjuvants and their concentrations must be such that they do not modify the property desired for the emulsion of the invention.

Mention may be made, as fillers which can be used in the composition of the invention, for example, of pigments, such as titanium, zinc or iron oxides and organic pigments; kaolin; silica; talc; boron nitride; spherical organic powders; fibres; and their mixtures. Mention may be made, as spherical organic powders, for example, of polyamide powders and in particular Nylon®, such as Nylon-1 or Polyamide 12, powders sold under the Orgasol names by Atochem; polyethylene powders; Teflon®; microspheres based on acrylic copolymers, such as those made of ethylene glycol dimethacrylate/lauryl methacrylate copolymer sold by Dow Corning under the name Polytrap; expanded powders, such as hollow microspheres and in particular the microspheres sold under the name Expancel by Kemanord Plast or under the name Micropearl F 80 ED by Matsumoto; silicone resin microbeads, such as those sold under the name Tospearl by Toshiba Silicone; polymethyl methacrylate microspheres, sold under the name Microsphere M-100 by Matsumoto or under the name Covabead LH85 by Wackherr; ethylene/acrylate copolymer powders, such as those sold under the name Flobeads by Sumitomo Seika Chemicals; or powders formed from natural organic materials, such as starch powders, in particular powders formed from crosslinked or noncrosslinked maize, wheat or rice starches, such as the powders formed from starch crosslinked with octenyl succinic anhydride sold under the name Dry-Flo by National Starch. Mention may be made, as fibres, for example, of polyamide fibres, such as in particular fibres formed from Nylon 6 (or Polyamide 6) (INCI name: Nylon 6) or from Nylon 6,6 (or Polyamide 66) (INCI name: Nylon 66) or such as fibres formed from poly(p-phenylene terephthalamide); and their mixtures. These fillers can be present in amounts ranging from 0 to 20% by weight and preferably from 0.5 to 10% by weight, with respect to the total weight of the composition.

According to a specific embodiment of the invention, the composition according to the invention comprises, as fillers, exfoliating particles which will make possible scrubbing of the skin. Use may be made, as exfoliating particles, of exfoliating or scrubbing particles of mineral, vegetable or organic origin. Thus, use may be made, for example, of polyethylene beads or powder, such as those sold under the name Microthene MN 727 or Microthene MN 710-20 by Equistar or such as the powder sold under the name Gotalene 120 Colorless 2 by Dupont; Nylon particles, such as those sold by Arkema under the name Orgasol 2002 EXD NAT COS; fibres, such as polyamide fibres, such as those sold by Utexbel under the name Pulpe Polyamide 12185 Taille 0.3 mm; poly(vinyl chloride) powder; pumice (INCI name) such as pumice 3/B from Eyraud; ground fruit kernel shells, such as ground materials derived from apricot kernels or walnut shells; sawdust; glass beads; alumina (INCI name) (aluminium oxide), such as the product sold under the name Dermagrain 900 by Marketech International; sugar crystals; beads which melt during application to the skin, such as, for example, spheres based on mannitol and cellulose which are sold under the Unisphere names by Induchem, agar-based capsules which are sold under the Primasponge names by Cognis and spheres based on joboba esters which are sold under the Florasphere names by Floratech; and their mixtures.

Mention may be made, as active principles which can be used in the composition of the invention, for example, of moisturizing agents, such as protein hydrolysates; sodium hyaluronate; polyols, such as glycerol, glycols, such as polyethylene glycols, and sugar derivatives; anti-inflammatories; procyanidol oligomers; vitamins, such as vitamin A (retinol), vitamin E (tocopherol), vitamin K, vitamin C (ascorbic acid), vitamin B5 (panthenol), vitamin B3 or PP (niacinamide), the derivatives of these vitamins (in particular esters) and their mixtures; keratolytic and/or desquamating agents, such as salicylic acid and its derivatives, α-hydroxy acids, such as lactic acid and glycolic acid, and their derivatives, and ascorbic acid and its derivatives; urea; caffeine; depigmenting agents, such as kojic acid, hydroquinone and caffeic acid; salicylic acid and its derivatives; retinoids, such as carotenoids and vitamin A derivatives; hydrocortisone; melatonin; algal, fungal, plant, yeast or bacterial extracts; steroids; antibacterial active principles, such as 2,4,4′-trichloro-2′-hydroxydiphenyl ether (or triclosan), 3,4,4′-trichlorocarbanilide (or triclocarban) and the acids indicated above and in particular salicylic acid and its derivatives; enzymes; flavonoids; tightening agents, such as synthetic polymers, plant proteins, polysaccharides of plant origin in or not in the form of microgels, starches, wax dispersions, mixed silicates and colloidal particles of inorganic fillers; ceramides; anti-inflammatory agents; soothing agents; mattifying agents; agents for combating hair loss and/or for regrowth of the hair; anti-wrinkle agents; essential oils; and their mixtures; and any active principle appropriate for the final objective of the composition.

The UV screening agents can be organic or inorganic (or physical UV screening agents). They can be present in an amount, as active material, ranging from 0.01 to 20% by weight of active material, preferably from 0.1 to 15% by weight and better still from 0.2 to 10% by weight, with respect to the total weight of the composition.

Mention may be made, as examples of organic screening agents active in the UV-A and/or UV-B regions which can be added to the composition of the invention, for example of anthranilates; cinnamic derivatives; dibenzoylmethane derivatives; salicylic derivatives, camphor derivatives; triazine derivatives, such as those described in patent applications U.S. Pat. No. 4,367,390, EP 863 145, EP 517 104, EP 570 838, EP 796 851, EP 775 698, EP 878 469 and EP 933 376; benzophenone derivatives; β,β-diphenylacrylate derivatives; benzotriazole derivatives; benzimidazole derivatives; imidazolines; bis-benzoxazolyl derivatives; such as described in Patents EP 669 323 and U.S. Pat. No. 2,463,264; p-aminobenzoic acid (PABA) derivatives; methylenebis[(hydroxyphenyl)benzotriazole] derivatives, such as described in applications U.S. Pat. No. 5,237,071, U.S. Pat. No. 5,166,355, GB 2 303 549, DE 19726184 and EP 893 119; screening polymers and screening silicones, such as those described in particular in Application WO 93/04665; or dimers derived from a-alkylstyrene, such as those described in Patent Application DE 19855649.

The total amount of organic UV screening agents in the compositions according to the invention can range, for example, from 0.1 to 20% by weight, with respect to the total weight of the composition, preferably ranging from 0.2 to 15% by weight, with respect to the total weight of the composition.

Mention may be made, as physical screening agents which can be added to the composition of the invention, for example, of coated or uncoated metal oxide pigments and nanopigments, in particular titanium, iron, zirconium, zinc or cerium oxides and their mixtures, it being possible for these oxides to be in the form of optionally coated micro- or nanoparticles (nanopigments).

The compositions which are subject-matters of the invention are intended for a topical application and can constitute in particular a dermatological or cosmetic composition, for example intended for caring for (anti-wrinkle, anti-ageing, moisturizing, anti-sun protection, and the like), for treating, for cleaning and for making up keratinous substances and in particular the skin, lips, hair, eyelashes and nails of human beings.

Substrate

The water-insoluble substrate can comprise one or more layers and it can be chosen from the group consisting of woven materials, non-woven materials, foams, sponges, waddings, as sheets, balls or films. It can in particular be a non-woven substrate based on fibres of natural origin (flax, wool, cotton, silk, fibres made of bamboo) or synthetic origin (cellulose derivatives, viscose, polyvinyl derivatives, polyesters, such as poly(ethylene terephthalate), polyolefins, such as polyethylene (PET) or polypropylene, polyamides, such as Nylon, or acrylic derivatives) and their mixtures, such as viscose/PET, polylactic acid (PLA) or viscose/polylactic acid (viscose/PLA).

According to a preferred embodiment of the invention, the substrate is a non-woven material. A general description of non-woven materials is given in Riedel “Non-woven Bonding Methods and Materials”, Non-woven World (1987). These substrates are obtained according to the normal methods of the technology for the preparation of non-woven materials.

When the substrate is a non-woven material, use is preferably made of a non-woven material which does not go into ball and which is sturdy enough not to disintegrate and not to become fluffy when applied to the skin. It must be absorbent and soft at least on one side for the removal of makeup from the eyes in particular. Mention may be made, as appropriate non-woven materials, for example, of those sold under the names Ultraloft 15285-01, Ultraloft 182-008, Ultraloft 182-010 and Ultraloft 182-016 by BBA, Vilmed M1519 Blau, Vilmed M 1550 N and 112-132-3 by Freudenberg, that sold under the name Norafin 11601-010B by Jacob Holm Industries, the flocked non-woven materials sold under the names Univel 109 and Univel 119 by Uni Flockage and that made of viscose/PLA supplied by Sandler.

According to one embodiment, the substrate is a non-woven material, 1000 composed of viscose or a non-woven material composed of a viscose/polylactic acid (viscose/PLA) mixture.

This substrate can comprise one or more layers having identical or different properties and having properties of elasticity and of softness and other properties appropriate to the desired use. The substrates can comprise, for example, two parts having different elasticity properties, as described in the document WO-A-99/13861, or can comprise a single layer having different densities, as described in the document WO-A-99/25318, or can comprise two layers of different textures, as described in the document WO-A-98/18441.

In addition, when the article is used for the body, the substrate can comprise at least one rough side for making it possible, at the same time, to massage the skin or to scrub the skin.

As indicated above, the substrate can have any size and any shape which are appropriate for the desired objective. Furthermore, it generally has a surface area of between 0.005 m² and 0.1 m², preferably between 0.01 m² and 0.05 m².

The degree of impregnation of the composition onto the substrate generally ranges from 100 to 1000%, preferably from 250 to 700%, of the weight of the substrate. The techniques for impregnating the substrates with compositions are well known in this field and can all be applied in the present invention. Generally, the impregnating composition is heated and added to the substrate by one or more techniques comprising immersion, coating, spraying, and the like.

Another subject-matter of the invention is a cosmetic method for caring for the skin or cleaning and/or removing makeup from the skin which consists in applying, to the skin, an article as defined above.

The examples below according to the invention are given by way of illustration and without a limiting nature. The names are the chemical name or the INCI name. The amounts are given therein as % by weight, unless otherwise mentioned.

### Example 1

### Makeup-Removing O/W Emulsion According to the Invention

The following Emulsion 1 according to the invention is prepared:

| | | |
|---|---|---|
| Phase A | Water | 40 |
| Phase B | Ethylhexyl palmitate | 2 |
| | Acrylate/C10-C30 alkyl acrylate | 0.1 |
| | copolymer (Pemulen TR-2 from | |
| | Lubrizol) | |
| Phase C | Ethylhexyl palmitate | 18 |
| | Preservatives | q.s. |
| Phase D | Water | 40 |
| | Lauryl inulin carbamate | 0.2 |
| | comprising 96.5% by weight of | |
| | active material (Inutec SP1 from | |
| | Orafti) | |
| Phase E | Water | q.s. |
| | | for 100 |
| Phase F | Sodium hydroxide | 0.04 |

Procedure:

The water (phase A) is heated to 60° C.

Phase B is incorporated with stirring until the gelling agent has completely dispersed.

Phase C is added with stirring.

Phase D is added with stirring until the inulin has completely dispersed.

Phase E is added and then the mixture is allowed to return to ambient temperature.

The Pemulen is neutralized by adding phase F.

The composition presents a viscosity, measured with the spindle 2 according to the method indicated above, of 80 mPa·s.

### Comparative Examples 2 and 3

The following emulsions are prepared with gelling acrylate copolymer and without hydrophobic modified inulin:

| | | Example 2 | Example 3 |
|---|---|---|---|
| | | (comparative) % | (comparative) % |
| Phase A | Water | 40 | 40 |
| Phase B | Ethylhexyl | 2 | 2 |
| | palmitate | | |
| | Acrylate/C10-C30 | 0.1 | 0.2 |
| | alkyl acrylate | | |
| | copolymer | | |
| | (Pemulen TR-2 | | |
| | from Lubrizol) | | |
| Phase C | Ethylhexyl | 18 | 18 |
| | palmitate | | |
| | Preservatives | q.s | q.s |
| Phase D | Water | q.s. for 100 | q.s. for 100 |
| Phase E | Sodium hydroxide | 0.04 | 0.04 |

### Procedure:

Phase A is heated to 60° C.

Phase B is incorporated with stirring until the gelling agent has completely dispersed.

Phase C is incorporated with stirring in order to form the emulsion.

Phase D is added in order to bring the preparation back to ambient temperature.

The Pemulen is neutralized with addition of phase E.

The composition of Example 2 exhibits phase separation after 2 months at 45° C.

The composition of Example 3 is stable but exhibits a viscosity, measured according to the method indicated above (spindle 2), of 150 mPa·s, which does not make possible good impregnation on a wipe.

### Comparative Example 4

The following emulsions are prepared with hydrophobic modified inulin and without gelling acrylate copolymer:

| | |
|---|---|
| Lauryl inulin carbamate comprising 96.5% | 0.2 |
| by weight of active material (Inutec SP1 | |
| from Orafti) | |
| Ethylhexyl palmitate | 20 |
| Preservatives | q.s. |
| Water | q.s. for |
| | 100 |

The water is heated to 60° C. and then the remaining compounds are incorporated with stirring until the emulsion has been formed.

The preparation is allowed to cool until it has returned to ambient temperature.

The composition of Example 4 separates into phases from 24 h after manufacture.

These examples show that the combination of a gelling polymer according to the invention and of hydrophobic modified inulin makes it possible to obtain a stable emulsion with a viscosity such that it makes possible homogeneous impregnation on an insoluble substrate, such as a wipe.

### Measurement of the Makeup-Removing Power

The makeup-removing power of:
a wipe A (nonwoven support, 100% viscose, 50 g/m²) 400% impregnated with composition 1 according to the invention) and
a wipe B, available commercially, comprising a low level of oil (nonwoven support, 100% viscose, 50 g/m²), 400% impregnated with the following composition:

| | |
|---|---|
| Triethanolamine | 0.1 |
| Disodium cocoamphodiacetate (Miranol C2M | 0.15 |
| Conc. NP from Rhodia) | |
| Cetyl alcohol | 0.15 |
| Isopropyl palmitate | 0.9 |
| Preservatives | q.s. |
| Carbomer (Carbopol 981 polymer from | 0.1 |
| Lubrizol) | |
| Xanthan gum | 0.05 |
| 1,2-Pentanediol | 0.5 |
| Glycerol | 3 |
| EDTA | 0.1 |
| Glyceryl mono/distearate/polyethylene | 0.56 |
| glycol (with 100 EO) stearate mixture | |
| (Arlacel 165 FL from Croda) | |
| Water | q.s. for 100 |
| was determined according to the protocol described | |
| below. | |

### 1. Materials/Equipment

Makeup-removing wipes A and B
Household soap
1 masking card cut out of a Bristol board of 4×4 cm+1 Lumicolor permanent S felt-tipped pen
—CR300 colorimeter (measurement of the L.a.b colorimetry)
Air Wear LSF 12 transfer-free foundation
1 stopwatch

### 2. Protocol

a—Mark out 4 areas on the skin of the arms (2 per arm) using the felt-tipped pen and the masking card
b—Clean the skin with the soap, rinse and dry (the marks from the felt-tipped pen are then toned down so as to prevent any transfer of ink from the felt-tipped pen into the foundation at the moment of application)
c—Measure the bare skin by colorimetry: 3 L,a,b measurements per area
d—Evenly apply 16 mg+/−0.05 mg of foundation in the 4 delimited areas and wait 30 minutes
e—Measure the skin with foundation by colorimetry: 3 L,a,b measurements per area
f—Remove makeup from the area with the wipe by a circular movement for 10 seconds and then rinse with tap water (warm; hardness not controlled) while lightly brushing with the fingers of the available hand. Do this to the 4 areas without waiting.
g—Dab with a paper handkerchief of Kleenex type and wait 15 minutes (feeling of dry skin)
h—Measure by colorimetry the skin from which the makeup has been removed: 3 L,a,b measurements per area

### 3. Calculation of the Percentage of Removal of Makeup

a—Colorimetric difference between the madeup skin and the bare skin=ΔEmax:

ΔEmax=\sqrt{(Δa₁²+Δb₁²+ΔL₁²)} with
Δa₁=a bare skin−a foundation skin
Δb₁=b bare skin−b foundation skin
ΔL₁=L bare skin−L foundation skin
b—For a makeup-removing product: colorimetric difference between the skin from which the makeup has been removed (remove skin) and the madeup skin=ΔE for each area:

ΔE=\sqrt{(Δa₂²+Δb₂²+ΔL₂²)} with
Δa₂=a remove skin−a foundation skin
Δb₂=b remove skin−b foundation skin
ΔL₂=L remove skin−L foundation skin

The mean percentage of removal of makeup corresponds to the mean of the 4 values of (ΔE/ΔEmax)*100 for a given makeup-removing product.

The results of the percentage of removal of makeup (percentage of makeup removed) for each composition are given in the table below.

| | Percentage of removal of |
|---|---|
| Composition | makeup |
| Wipe A (invention) | 54% |
| Wipe B (comparative) | 18% |

This table shows that the composition according to the invention is much more effective in removing makeup than the composition of the comparative example.

### Example 5

### Makeup-Removing O/W Emulsion According to the Invention

The following Emulsion 5 according to the invention is prepared:

| | | |
|---|---|---|
| Phase A | Water | 40 |
| Phase B | Sodium 2-acryl-amido-2-methyl- | 0.5 |
| | propanesulphonate/hydroxyethyl | |
| | acrylate copolymer as a 40% | |
| | inverse emulsion in | |
| | Polysorbate 60 and squalane | |
| | (Simulgel NS from Seppic | |
| Phase C | Lauryl inulin carbamate | 0.2 |
| | comprising 96.5% by weight of | |
| | active material (Inutec SP1 | |
| | from Orafti) | |
| Phase D | Ethylhexyl palmitate | 200 |
| | Preservatives | q.s. |
| Phase E | Water | q.s. for 100 |

### Procedure:

The water (phase A is heated to 60° C.

Phase B is incorporated with stirring until the gelling agent has completely dispersed.

Phase C is added with stirring until the inulin has completely dispersed.

The compounds of phase D are mixed and heated to 60° C., and then phase D is added to the mixture (A+B+C) with stirring.

Phase E is added and then the mixture is allowed to return to ambient temperature.

The composition exhibits a viscosity measured with the spindle 2 according to the method indicated above, of 90 mPa·s

### Comparative Examples 6 and 7

The following emulsions are prepared with AMPS copolymer and without hydrophobic modified inulin:

| | | Example 6 | Example 7 |
|---|---|---|---|
| | | (comparative) | (comparative) |
| | | % | % |
| Phase | Water | 40 | 40 |
| A | | | |
| Phase | Ethylhexyl palmitate | 20 | 20 |
| B | Sodium 2-acrylamido-2-methyl- | 0.6 | 0.7 |
| | propanesulphonate/hydroxyethyl | | |
| | acrylate copolymer as a 40% | | |
| | inverse emulsion in | | |
| | Polysorbate 60 and squalane | | |
| | (Simulgel NS from Seppic) | | |
| | Preservatives | q.s. | q.s. |
| Phase | Water | q.s. for | q.s. for |
| C | | 100 | 100 |

Procedure:

Phase A is heated to 60° C.

Phase B is incorporated with stirring until the gelling agent has completely dispersed.

Phase C is added in order to bring the preparation back to ambient temperature.

The composition of Example 6 exhibits phase separation 24 hours after manufacture.

The composition of Example 7 is stable but exhibits a viscosity, measured according to the method indicated above (spindle 2), of 130 mPa·s, which does not make possible good impregnation on a wipe.

### Comparative Example 8

The following emulsion is prepared with hydrophobic modified inulin and without gelling AMPS copolymer:

| | |
|---|---|
| Lauryl inulin carbamate comprising | 0.2 |
| 96.5% by weight of active material | |
| (Inutec SP1 from Orafti) | |
| Ethylhexyl palmitate | 20 |
| Preservatives | q.s. |
| Water | q.s. for 100 |

The water is heated to 60° C. and then the remaining compounds are incorporated with stirring until the emulsion has been formed.

The preparation is allowed to cool until it has returned to ambient temperature.

The composition of Example 8 separates into phases from 24 h after manufacture.

These examples show that the combination of a gelling polymer according to the invention and of hydrophobic modified inulin makes it possible to obtain a stable emulsion with a viscosity such that it makes possible homogeneous impregnation on an insoluble substrate, such as a wipe.

The above written description of the invention provides a manner and process of making and using it such that any person skilled in this art is enabled to make and use the same, this enablement being provided in particular for the subject matter of the appended claims, which make up a part of the original description.

As used herein, the words “a” and “an” and the like carry the meaning of “one or more.”

The phrases “selected from the group consisting of,” “chosen from,” and the like include mixtures of the specified materials. Terms such as “contain(s)” and the like are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. An article comprising:
(A) a water-insoluble substrate comprising one or more layers, and
(B) a composition added to, in contact with, or impregnated on the substrate, in the form of an oil-in-water emulsion comprising an oily phase dispersed in an aqueous phase, comprising:
at least one thickening polymer chosen from i) copolymers resulting from the polymerization of at least one monomer (a) chosen from carboxylic acids possessing α,β-ethylenic unsaturation or their esters with at least one monomer (b) possessing ethylenic unsaturation comprising a hydrophobic group, ii) polymers comprising at least one monomer possessing a sulpho group and their mixtures, and
at least one inulin modified by hydrophobic chains.

2. The article according to claim 1, wherein the inulin modified by hydrophobic chains represents 0.01 to 20% by weight with respect to the total weight of the composition.

3. The article according to claim 1, wherein the inulin modified by hydrophobic chains is obtained from chicory.

4. The article according to claim 1, wherein the hydrophobic chains of the modified inulin are laurylcarbamate groups.

5. The article according to claim 1, wherein the thickening polymer represents 0.01 to 10% by weight with respect to the total weight of the composition.

6. The article according to claim 1, wherein the hydrophobic group of the thickening copolymer i) resulting from the polymerization of at least one monomer (a) and at least one monomer (b) comprises at least 8 carbon atoms.

7. The article according to claim 1, wherein the thickening polymer is chosen from copolymers resulting from the polymerization:
of at least one monomer of following formula (1): 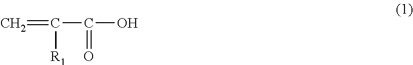
in which R₁ denotes H or CH₃ or C₂H₅, and
of at least one monomer of unsaturated carboxylic acid (C₁₀-C₃₀)alkyl ester type which corresponds to the monomer of following formula (2): 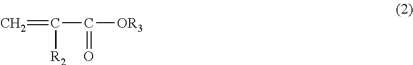
in which R₂ denotes H or CH₃ or C₂H₅ and R₃ denotes a C₁₀-C₃₀ alkyl radical.

8. The article according to claim 7, wherein the monomers of formula (2) are chosen from lauryl acrylate, stearyl acrylate, decyl acrylate, isodecyl acrylate, dodecyl acrylate, lauryl methacrylate, stearyl methacrylate, decyl methacrylate, isodecyl methacrylate, dodecyl methacrylate, and their mixtures.

9. The article according to claim 1, wherein the thickening polymer is a crosslinked polymer.

10. The article according to claim 1, wherein the thickening polymer is chosen from polymers formed from a mixture of monomers comprising:
(i) acrylic acid,
(ii) an unsaturated carboxylic acid (C₁₀-C₃₀)alkyl ester corresponding to the monomer of following formula (2): 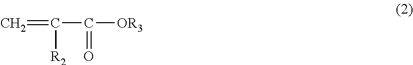
in which R₂ denotes H or CH₃ and R₃ denotes an alkyl radical having from 12 to 22 carbon atoms,
(iii) and a crosslinking agent which is a copolymerizable polyethylenic unsaturated monomer.

11. The article according to claim 1, wherein the thickening polymer is chosen from copolymers comprising 95 to 60% by weight of acrylic acid, 4 to 40% by weight of C₁₀-C₃₀ alkyl acrylate and 0 to 6% by weight of crosslinking polymerizable monomer; copolymers comprising 98 to 96% by weight of acrylic acid (hydrophilic unit), 1 to 4% by weight of C₁₀-C₃₀ alkyl acrylate (hydrophobic unit) and 0.1 to 0.6% by weight of crosslinking polymerizable monomer; and their mixtures.

12. The article according to claim 1, wherein the polymer comprising at least one monomer possessing a sulpho group is chosen from 2-acrylamido-2-methylpropanesulphonic acid (AMPS) copolymers.

13. The article according to claim 1, wherein the polymer comprising at least one monomer possessing a sulpho group is chosen from copolymers of (meth)acrylic acid or of (meth)acrylate and of 2-acrylamido-2-methylpropanesulphonic acid.

14. The article according to claim 1, wherein the polymer comprising at least one monomer possessing a sulpho group is chosen from copolymers of hydroxyalkyl acrylate and of AMPS.

15. The article according to claim 1, wherein the hydrophobic modified inulin/thickening polymer ratio by weight is 5:1 to 1:5.

16. The article according to claim 1, wherein the oily phase comprises at least one oil chosen from isopropyl myristate, isopropyl palmitate, 2-ethylhexyl caprate/caprylate, 2-ethylhexyl palmitate, isostearyl neopentanoate, isononyl isononanoate, hexyl laurate, esters of lactic acid and of fatty alcohols comprising 12 or 13 carbon atoms, dicaprylyl carbonate, dicaprylyl ether, the 2-ethylhexyl ether of glycerol and their mixtures.

17. The article according to claim 1, wherein the composition comprises at least 1% of oil with respect to the total weight of the composition.

18. A composition in the form of an oil-in-water emulsion comprising an oily phase dispersed in an aqueous phase, comprising:
at least one thickening polymer chosen from i) copolymers resulting from the polymerization of at least one monomer (a) chosen from carboxylic acids possessing α,β-ethylenic unsaturation or their esters with a monomer (b) possessing ethylenic unsaturation comprising a hydrophobic group, ii) polymers comprising at least one monomer possessing a sulpho group and their mixtures, and
at least one inulin modified by hydrophobic chains,
the aqueous phase being present in a content of greater than or equal to 40% by weight with respect to the total weight of the composition, the composition comprising less than 5% by weight of additional emulsifying agents, with respect to the total weight of the composition.

19. A method for caring for the skin or cleaning and/or removing makeup from the skin comprising contacting the skin with an article according to claim 1.
